(19) United States
(12) Patent Application Publication
(10) Pub. No.: US 2011/0087153 A1
(43) Pub. Date: Apr. 14, 2011

(54) Transdermal Methods And Systems For The Delivery Of Rizatriptan

(76) Inventors: Angel S. ANGELOV, Philadelphia, PA (US); David B. LEBO, Philadelphia, PA (US)

(21) Appl. No.: 12/902,837

(22) Filed: Oct. 12, 2010

Related U.S. Application Data
(60) Provisional application No. 61/250,933, filed on Oct. 13, 2009.

(51) Int. Cl. A61K 31/4196; A61K 9/00; A61P 25/06; A61N 1/30

(52) U.S. Cl. 604/20; 424/484; 514/383

(57) ABSTRACT

Iontophoretic patches for the delivery of rizatriptan and methods of using the patches are described.

### RELATED APPLICATIONS

The present invention is related and claims priority to U.S. Provisional Application Ser. No. 61/250,933, filed Oct. 13, 2009. The entire contents of this application are expressly incorporated herein by this reference.

### BACKGROUND OF THE INVENTION

The process of iontophoresis was described by LeDuc in 1908 and has since found commercial use in the delivery of ionically charged therapeutic agent molecules such as pilocarpine, lidocaine and dexamethasone. In this delivery method, ions are carried into the skin by an electrical current that is supplied by a positive and negative electrode. Positive ions are carried away from the positive anode, while negative ions are carried away from the negative cathode.

Earlier, and some present, iontophoretic devices were typically constructed of two electrodes attached by adhesive materials to a patient, each connected by a wire to a remote power supply. These devices were generally microprocessor-controlled electrical instruments.

More recently, self-contained wearable iontophoretic systems have been developed. These systems are advantageous in that they do not have external wires and are much smaller in size. Examples of such systems can be found in a variety of U.S. patents, including U.S. Pat. Nos. 4,927,408; 5,358,483; 5,458,569; 5,466,217; 5,533,971; 5,605,536; 5,651,768; 5,685,837; 6,421,561; 6,425,892; 6,653,014; and 6,745,071. These systems are also comprised of two electrodes fixed to patients by means of adhesive materials.

### SUMMARY OF THE INVENTION

Despite the above, there exists the need for an optimal product that would provide the advantages of rapid, systemic rizatriptan administration with a consistent duration of action.

The invention pertains, at least in part, to a method for treating a subject for a rizatriptan responsive state, comprising transdermally administering to the subject an effective amount of rizatriptan or a salt thereof in less than 45 minutes using an integrated iontophoretic patch, wherein the rizatriptan or salt thereof is formulated in a flowable hydrogel and wherein said patch uses a current density selected such that said current does not substantially irritate said subject's skin.

In another embodiment, the invention includes an integrated iontophoretic transdermal patch for the delivery of rizatriptan or a salt thereof to a subject in need thereof, comprising a controller to deliver at least a portion of said rizatriptan or salt thereof to the subject by driving an electrotransport current through an animal body surface using a controllable power supply; an electrode which does not form an insoluble salt of the rizatriptan or salt thereof; and a composition comprising rizatriptan or a salt thereof formulated in a flowable hydrogel; wherein the patch delivers an effective amount of rizatriptan or a salt thereof to the subject in less than 45 minutes.

In some embodiments, an effective amount of rizatriptan or a salt thereof is administered to the subject in less than 30 minutes. In some embodiments, the effective amount is a concentration of about 20 ng/mL or less in the subject's blood. In some embodiments, the patch is able to maintain an effective steady state concentration of the rizatriptan or salt thereof in the subject's blood for at least about an hour.

In some embodiments, the patch provides rizatriptan at an iontophoretic flux rate of about 22.9 mcg/cm²/min or greater across the subject's skin, e.g., an iontophoretic flux rate of about 28.5 mcg/cm²/min or greater across the subject's skin.

In some embodiments, the patch uses an average current density of 0.20 mA/cm² or less, e.g., an average current density of 0.15 mA/cm² or less, e.g., an average current density of 0.10 mA/cm² or less, e.g., an average current density of 0.05 mA/cm² or less for a significant portion of delivery time of the rizatriptan or salt thereof.

In some embodiments, the patch (e.g., the controller) provides an uninterrupted two-stage patterned delivery sequence wherein current densities average between about 0.05 and about 0.40 mA/cm² during a significant portion of a first stage followed by a second stage delivery wherein current densities average between about 0.01 and about 0.40 mA/cm² during a significant portion of the second stage, to provide a waveform delivery pattern in which a therapeutically effective dosage level is reached in a subject in less than about one hour and a maintenance level is continued for one or more hours.

In some embodiments, usage of the patch to deliver a steady state concentration of rizatriptan results in a mean skin erythema score of 1.00 or less, e.g., 0.50 or less, e.g., zero, immediately after patch removal.

In some embodiments, the hydrogel comprises a flux enhancer, such as a flux enhancer which is uncharged at neutral pH. Flux enhancers can be, for example lauric acid, polyoxyethylene (4) lauryl ether and mixtures thereof. In some embodiments, the hydrogel includes at least about 3.4% polyoxyethylene (4) lauryl ether. In some embodiments, the patch includes at least 4% rizatriptan and at least 3.4% polyoxyethylene (4) lauryl ether.

### BRIEF DESCRIPTION OF THE DRAWINGS

FIG. 1 is a graph depicting data regarding permeation of Sumatriptan through the dialysis membrane.

FIG. 2 is a graph depicting both the iontophoresis of rizatriptan and the reversed-electrodes iontophoresis data.

FIG. 3 is a graph depicting the permeation data for Zolmitriptan.

FIG. 4 is a graph depicting the HPLC results for the analysis of rizatriptan through the dialysis membrane.

FIG. 5 is a graph showing permeation of migraine candidates through human cadaver skin.

FIG. 6 is a graph showing permeation of migraine candidates formulated in a gel through human cadaver skin.

FIG. 7 is a graph showing iontophoresis of migraine candidate drugs in formulated products.

### DETAILED DESCRIPTION

The present invention is based, at least in part, on the discovery that rizatriptan has a superior (e.g., greater) flux through human cadaver skin than other triptan compounds. Without wishing to be bound by any particular theory, it is believed that rizatriptan may be formulated into an iontophoretic patch which has superior properties to patches which include other triptan compound. For example, patches of the present invention have, in some embodiments, a very low current density, and/or a small surface area. Additionally, in some embodiments, patches of the present invention are capable of delivering rizatriptan quickly (e.g., less than about 30 minutes) and are capable of maintaining the level of rizatriptan in the blood over an extended period of time (e.g., more than an hour).

It is also believed, without being bound by theory, that the superior flux allows for the administration of a therapeutically effective amount of rizatriptan without substantial skin erythema (e.g., with a mean erythema score of less than about 1).

An additional advantage of the methods of the present invention over oral administration of rizatriptan is that there is less variation of pharmacokinetic parameters with the present invention as compared to oral delivery.

1. Methods of Treating Subjects Using the Patches of the Invention

In some embodiments, the invention pertains to a method for treating a subject for a rizatriptan responsive state. The method includes transdermally administering to the subject an effective amount of rizatriptan or a salt thereof in less than 45 minutes using an integrated iontophoretic patch. In some embodiments, the rizatriptan or salt thereof is formulated in a flowable hydrogel. In some embodiments, the patch uses a current density selected such that said current does not substantially irritate said subject's skin.

In some embodiments, the patch of the invention allows for the delivery of an effective amount of rizatriptan or a salt thereof to a subject in less than about 40 minutes. In some embodiments, the patch of the invention allows for the delivery of an effective amount of rizatriptan or a salt thereof to a subject in less than about 35 minutes. In some embodiments, the patch of the invention allows for the delivery of an effective amount of rizatriptan or a salt thereof to a subject in less than about 30 minutes. In some embodiments, the patch of the invention allows for the delivery of an effective amount of rizatriptan or a salt thereof to a subject in less than about 25 minutes. In some embodiments, the patch of the invention allows for the delivery of an effective amount of rizatriptan or a salt thereof to a subject in less than about 20 minutes. In some embodiments, the patch of the invention allows for the delivery of an effective amount of rizatriptan or a salt thereof to a subject in less than about 15 minutes. In some embodiments, the patch of the invention allows for the delivery of an effective amount of rizatriptan or a salt thereof to a subject in less than about 10 minutes.

In some embodiments, the patch provides a beneficial flux rate across the subject's skin. For example, the flux rate of rizatriptan across the subject's skin can be greater than about 20 mcg/cm²/min, greater than about 21 mcg/cm²/min, greater than about 22 mcg/cm²/min, greater than about 23 mcg/cm²/min, greater than about 24 mcg/cm²/min, greater than about 25 mcg/cm²/min, greater than about 26 mcg/cm²/min, greater than about 27 mcg/cm²/min, greater than about 28 mcg/cm²/min, greater than about 29 mcg/cm²/min, greater than about 30 mcg/cm²/min, or more. In some embodiments, the patch provides rizatriptan at an iontophoretic flux rate of about 22.9 mcg/cm²/min or greater across the subject's skin. In some embodiments, the patch provides rizatriptan at an iontophoretic flux rate of about 28.5 mcg/cm²/min or greater across the subject's skin.

The term “subject” includes living organisms capable of having rizatriptan responsive states (e.g., mammals). Examples of subjects include humans, dogs, cats, horses, cows, goats, rats and mice. In some embodiments, the subject is a human. In some embodiments, the term includes subjects suffering from a rizatriptan responsive state.

Examples of pharmaceutically acceptable salts of rizatriptan which may be used in the methods and patches of the invention include, but are not limited to, chloride, bromide, iodide, sulfuric, phosphate, benzoate, lactate, citrate, tartarate, salicylate, succinate, maleate, gluconate, mesylate, laurate, dodecylate, myristate, palmitate, stearate, coconoate, behinate, oleate, linoleate, linolenate, eicosapentaenoate, eicosahexaenoate, docosapentaenoate, docosahexaenoate, eicosanoids and the like. In some embodiments, the rizatriptan salt is rizatriptan benzoate. In some embodiments, the salt of rizatriptan is selected such that it does not react with the other components of the patch, such as the metal electrode. In some embodiments, the salt is selected such that it does not form a significant amount of an insoluble residue when in contact with the metal components of the patch of the invention.

The term “effective amount” includes the amount of the rizatriptan which is effective to treat a particular rizatriptan responsive state. In some embodiments, the effective amount of rizatriptan delivered systemically is greater than about 1.0 mg, greater than about 2.5 mg, greater than about 5 mg, greater than about 10 mg, or greater than about 15 mg. In some embodiments, the effective amount of rizatriptan may be a concentration of about 5 ng/mL or greater, about 6 ng/mL or greater, about 7 ng/mL or greater, about 8 ng/mL or greater, about 9 ng/mL or greater, about 10 ng/mL or greater, about 11 ng/mL or greater, about 12 ng/mL or greater, about 13 ng/mL or greater, about 14 ng/mL or greater, about 15 ng/mL or greater, about 17.5 ng/mL or greater, about 20 ng/mL or greater, about 25 ng/mL or greater in the subject's plasma. In some embodiments, an effective amount of rizatriptan achieves a maximum plasma concentration (Cₘₐₓ) of up 20 ng/mL.

The term “treat” includes the reduction or amelioration of one or more symptoms of the rizatriptan responsive state. It also may include the prevention of the occurrence or reoccurrence of a rizatriptan responsive state.

The term “rizatriptan responsive state” refers to diseases, disorders and states which are amenable to treatment with rizatriptan. Rizatriptan responsive states include migraines, familiar hemiplegic migraines (with and without aura), chronic paroxysmal headaches, cluster headaches, migraine headaches, basilar migraines, and atypical headaches accompanied by autonomic symptoms. In some embodiments, the rizatriptan responsive state is a migraine.

Migraine headache is associated with inflamed and dilated blood vessels leading to severe unilateral pain that worsens with physical activity. (The International Classification of Headache Disorders. Cephalagia. 24, Supplement 1: (2004)) In addition to headache pain, migraine can be associated with a variety of other symptoms, including diarrhea, cold extremities, facial pallor, nausea, vomiting and sensitivity to external stimuli such as light, sounds or odors. Approximately one fifth of migraine patients experience an aura or visual symptoms, such as spots of light, zigzag lines, or a graying out of vision. (Cephalagia (2004) and J. Scholpp, R. Shellenberg, B. Moeckesch, and N. Banik. Migraines typically last for up to 24 hours, but can range from 4 to 72 hours and patients often experience migraine attacks one to two times per month.

The term “delivery” includes the transport of the rizatriptan from the patch to the plasma of a subject. In certain embodiments, approximately 1%-30% (or higher) of the rizatriptan in the patch is delivered to the plasma of the subject over the course of treatment.

The term “transdermal” includes delivery methods which occur through the skin of a subject without puncturing the subject's skin.

The term “delivery time” includes the period of time which the patch is functioning by actively delivering the rizatriptan to the subject iontophoretically.

In some embodiments, the patch is able to maintain a steady state concentration of the rizatriptan in the subject's plasma for at least one hour, for at least two hours, for at least three hours, for at least four hours, for at least five hours for at least six hours, for at least seven hours, or more.

In some embodiments, the plasma concentrations of rizatriptan are sustained at therapeutic levels for at least one hour. In some embodiments, the plasma concentrations of rizatriptan are sustained at therapeutic levels for at least two hours. In some embodiments, the plasma concentrations of rizatriptan are sustained at therapeutic levels for at least three hours. In some embodiments, the plasma concentrations of rizatriptan are sustained at therapeutic levels for at least four hours.

In some embodiments, the term “sustained” includes levels (e.g., plasma levels of rizatriptan) which fluctuate less than about 20%, less than about 10%, or less than about 5% over a period.

The language “maintain a steady state concentration” refers to the maintenance of a particular concentration (e.g., a desired concentration, e.g., an effective amount) for a particular length of time. In some embodiments, the concentration of the rizatriptan in the subject's plasma fluctuates from the average concentration by about 10 ng/ml or less, about 9 ng/ml or less, about 8 ng/ml or less, about 7 ng/ml or less, about 6 ng/ml or less, about 5 ng/ml or less, about 4 ng/ml or less, about 3 ng/ml or less, about 2 ng/ml or less, about 1 ng/ml or less, or by about 0.5 ng/ml or less.

In some embodiments, the present teachings provide methods of treating a subject for a rizatriptan responsive state using an iontophoretic patch as described herein, wherein the current does not substantially irritate the subject's skin. Without wishing to be bound by any particular theory, it is believed that current densities should be kept low in order to minimize skin irritation from current flow. At the same time, it is believed that the higher flux rate of the rizatriptan allows the current density to be maintained at a lower level. By means of the present invention, desired optimized dosage patterns are realized while maintaining safe current density levels thereby minimizing skin irritation effects. Accordingly, in some embodiments, current densities for the agents transported in the present invention are between 25 and 200 microamps per square centimeter, but may be as high as about 400 microamps per square centimeter in certain cases.

The language “does not substantially irritate a subject's skin” includes patches which result in a skin erythema score of 2.50 or less, 2.00 or less, or 1.00 or less about two hours, 24 hours, two days, three days, four days or one week after patch removal. In another further embodiment, the language “does not substantially irritate a subject's skin” includes patches which result in a skin erythema score of 2.50 or less, 2.00 or less, or 1.00 or less immediately after patch removal. In some embodiments, the patches of the invention do not cause punctuate lesions when used according to the methods of the invention.

In some embodiments, the patch employs a current which is effective to deliver the amount of rizatriptan or a salt thereof needed to treat the rizatriptan responsive state without substantially irritating the subject's skin. Without wishing to be bound by any particular theory, it is believed that the higher flux rate of rizatriptan (e.g., versus sumatriptan) also allows for the utilization of a lower current to deliver the rizatriptan in an effective amount. In some embodiments, the patch is uses a current greater than about 0.1 mA, greater than about 0.25 mA, greater than about 0.5 mA, greater than about 1 mA, greater than about 2 mA, greater than about 3 mA, greater than about 4 mA, or greater than about 5 mA, without substantially irritating a subject's skin. In some embodiments, the patch employs a current of about 4 mA for about an hour.

In some embodiments, the methods of the present invention include administering an effective amount of rizatriptan using an iontophoretic patch which utilizes a current density of at least about 0.10 mA/cm² or higher, 0.20 mA/cm² or higher, 0.30 mA/cm² or higher, or about 0.40 mA/cm² or higher for at least a portion of the treatment period. In some embodiments, the methods of the present invention include administering an effective amount of rizatriptan using an iontophoretic patch which utilizes a current density of about 0.25 mA/cm² or less, about 0.20 mA/cm² or less, about 0.10 mA/cm² or less. In some embodiments, the patch may use an average current density of 0.25 mA/cm² or less for a significant portion of the delivery time of the rizatriptan. The term “significant portion” includes at least 30% of the delivery time or more, at least 40% of the delivery time or more, at least 50% of the delivery time or more, at least 60% of the delivery time or more, at least 70% of the delivery time or more, at least 75% of the delivery time or more, at least 80% of the delivery time or more, at least 85% of the delivery time or more, at least 90% of the delivery time or more, or at least 95% of the delivery time or more.

In some embodiments, the invention also pertains, at least in part, to a method for treating a subject for a rizatriptan responsive state which includes administering to the subject an effective amount of rizatriptan, wherein the effective amount of rizatriptan is administered without substantial adverse effects.

The term “substantial adverse effects” includes those listed on current triptan product labels. Examples of these substantial adverse effects include atypical sensations (e.g., sensation of warmth or cold, parethesias, etc.) and pain and pressure sensations. Examples of adverse effects include, but are not limited to, mucosal burning sensations, ear discomfort, facial pain, feeling hot, flushing, head discomfort, hot flush, paraesthesia, sense of heaviness, sensation of pressure, neck pain, etc.

In some embodiments, the term “substantial adverse effects” do not include skin irritation or “application site disorders” caused by the patch itself.

In some embodiments, the methods of the present invention include administering an effective amount of rizatriptan using an iontophoretic patch which utilizes a single stage patterned delivery sequence, which may be uninterrupted and which may deliver rizatriptan at relatively high rate or at a lower rate to provide a waveform delivery pattern in which a therapeutically effective dosage level is reached quickly, e.g., less than 45 minutes, e.g., less than 30 minutes, and a maintenance level is continued, e.g., for one or more hours.

In some embodiments, the methods of the present invention include administering an effective amount of rizatriptan using an iontophoretic patch which utilizes a two-stage patterned delivery sequence, which may be uninterrupted and which may include an initial first stage delivery at a relatively high rate followed by a second stage delivery at a lower rate to provide a waveform delivery pattern in which a therapeutically effective dosage level is reached quickly, e.g., less than 45 minutes, e.g., less than 30 minutes, and a maintenance level is continued, e.g., for one or more hours.

In some embodiments, the methods of the present invention include administering an effective amount of rizatriptan using an iontophoretic patch which utilizes a multiple stage patterned delivery sequence, which may be uninterrupted and which may include multiple rates of delivery to provide a waveform delivery pattern in which a therapeutically effective dosage level is reached quickly, e.g., less than 45 minutes, e.g., less than 30 minutes, and a maintenance level is continued, e.g., for one or more hours.

The waveform pattern illustrates how a rapid onset of action can be created using a higher current, as well as how a sustained action can be obtained to improve the duration of action as compared to oral dosing. For example, although not to be limited by theory, it is believed that having the higher current density period first may provide relief for the subject for one or more the symptoms of the rizatriptan responsive state, e.g., migraine. The lower current density period may then prevent or delay the reoccurrence of the rizatriptan responsive state. The rapid peak level, followed by sustained lower maintenance level, is accomplished by a simple two step current pattern, and does not require an intermediate off cycle.

In some embodiments, the high current density period precedes the low current density period. Furthermore, in some embodiments, the current density of these periods are different, such that the current density of the high current density period is at least 10%, at least 20%, at least 30%, at least 40%, at least 50%, at least 60%, at least 70%, at least 80%, at least 90%, or at least 100% greater than the current density of the low current density period. In some embodiments, the current density of the high current density period is at least 2 times, at least 3 times, at least 4 times, or at least five times greater than the current density of the low current density period.

For example, in some embodiments, the first stage can average about 1.0 mA to about 3.0 mA. For example, in some embodiments, the first stage can average about 1.0 mA, about 1.5 mA, about 2.0 mA, about 2.5 mA or about 3.0 mA. In some embodiments, the duration of the first stage can average about 15 minutes to about 90 minutes. For example, in some embodiments, the duration of the first stage is about 15 minutes, about 30 minutes, about 45 minutes, about 60 minutes, about 75 minutes or about 90 minutes. The length and intensity of the higher current density period may be selected such that the subject may be treated for the acute symptoms of the rizatriptan responsive state. For example, the higher current density period may provide the subject with an effective dose of rizatriptan such that the primary symptoms of the rizatriptan responsive state, e.g., migraine, are eliminated or ameliorated.

In some embodiments, the second stage can average about 0.01 to about 2.0 mA. For example, in some embodiments, the second stage can average about 0.1 mA, about 0.25 mA, about 0.5 mA, about 1.0 mA, about 1.5 mA or about 2.0 mA. In some embodiments, the duration of the second stage can average about 45 minutes to about seven hours. For example, in some embodiments, the duration of the second stage is about 45 minutes, about 1 hour, about 2 hours, about 3 hours, about 4 hours, about 5 hours, about 6 hours or about 7 hours. The length and intensity of the lower current density period may be selected such that the subject may be treated for ongoing symptoms of the rizatriptan responsive state, or such that recurrences of acute symptoms are prevented.

In some embodiments, patches may be configured to employ other time-variable profiles involving multi-stage patterned deliveries of various rates and times and may include more than two stages. A multi-stage delivery that evidences a peak or crest in blood concentration levels may also be referred to as a “wave form pattern”.

One two-stage patterned delivery includes a first stage which averages approximately 3 mA for about 30 minutes, followed by a second stage which averages approximately 1.5 mA for at least 1 or more hours. Another two-stage patterned delivery includes a first stage which averages approximately 1 mA for about 45 minutes, followed by a second stage which averages approximately 0.5 mA for about 1 or more hours. In a further simulation of rizatriptan delivery, a current level of 3 mA is used for 45 minutes (first stage), followed by a sustained current of 1.5 mA for hours (second stage). In one embodiment, the first stage is about 30 minutes and the second stage is about 5.5 hours. These delivery pattern may be used for a transdermal patch that is superior in comparison to oral dosing.

The invention also provides multi-dosage rate devices that can provide waveform optimized dosage delivery patterns that can be used to provide minimal time for onset of action, while providing maximum time for therapeutic benefit thereafter. The present teachings provide this result using, for example, a simplified two-stage delivery pattern to create an initial peak dosage level, supplying an effective amount of anti-migraine agent in less than one hour, before falling to a lower sustained therapeutic level in a subject's blood.

The present teachings also provide the formulated content of rizatriptan in the iontophoretic delivery chambers required to maintain efficient delivery for the delivery period. For example, the amount of rizatriptan contained in the patch may be, e.g., at least twice, at least three fold, at least four fold, at least five fold, or at least six fold higher than the desired delivery dosage in order to provide a uniform delivery rate. In some embodiments, a formulation in accordance with the invention may also incorporate a hydrogel material. The hydrogel serves to retain the drug formulation in the reservoir, unlike free flowing liquids which are more prone to leakage during body movement. Further, the hydrogel can be provided as a very viscous fluid, which enables an optimal skin contact and minimizes skin irritation. An example of a viscous fluid for use in connection with the present teachings is a 2% hydroxypropylmethylcellulose (HPMC) or polyvinylpyrrolidinone (PVP).

Effective levels of rizatriptan in a subject's blood will vary with the severity of condition, etc. For this reason, a variety of patch dosages can be made available both as to amount and duration of sustained application.

In some embodiments, a device and delivery pattern is provided which supplies an optimal delivery rate for rizatriptan in order to provide an iontophoretic transdermal device for similar or superior efficacy to 10 mg oral delivery dosing forms. In some embodiments, a device and delivery pattern is provided which supplies an optimal delivery rate for rizatriptan in order to provide an iontophoretic transdermal device for similar or superior efficacy to 5 mg oral delivery dosing forms.

Although some embodiments utilize a two-stage delivery pattern to create an initial peak dosage level followed by a period of a lower sustained therapeutic level in a subject's blood, rizatriptan can also be administered to a subject utilizing a single stage. If this approach is used, however, the quick initial peak dosage level of administration may be replaced by a somewhat delayed sustained therapeutic level which may be advisable in some cases.

One aspect of the invention pertains to methods for delivering therapeutic rizatriptan using an iontophoretic transdermal patch which includes (a) providing an iontophoretic transdermal patch containing an amount of rizatriptan to be administered; (b) using a two-stage patterned delivery sequence, e.g., uninterrupted, which may include an initial first stage delivery at a relatively high rate followed by a second stage delivery at a lower rate to provide a waveform delivery pattern in which a therapeutically effective dosage level is reached in a subject in generally less than 45 minutes and a maintenance level is continued for one or more hours.

The invention pertains, at least in part, to an iontophoretic transdermal patch for the delivery of rizatriptan dedicated to a two-stage administration sequence in which an initial or first stage delivery rate exceeds a second stage delivery rate to produce a waveform delivery pattern. In some embodiments, the patch may be characterized by a power/area ratio such that the average current density is less than 250 μA/cm².

The invention also includes an integrated iontophoretic transdermal patch for the delivery of rizatriptan or a salt thereof. The patch allows for the delivery of an effective amount of rizatriptan to a subject in less than one hour, e.g., less than 45 minutes, less than 30 minutes, less than 25 minutes, less than 20 minutes, or even less than 15 minutes. In some embodiments, the patch is capable of maintaining a steady state concentration of rizatriptan in the subject at a desired concentration for at least one hour, e.g., at least 2, 3, 4 5, 6, 7, 8, 9, 10, 11, 12, 13, 14, 15, 16, 17, 18, 19, 20, 21, 22, 23 or even 24 hours. In some embodiments, the patch uses a current density selected such that the current does not substantially irritate the skin of a subject without damaged or denuded skin.

2. Iontophoretic Patches of the Invention

The invention pertains, at least in part, to an integrated iontophoretic transdermal patch for the delivery of rizatriptan or a salt thereof.

The terms “iontophoretic transdermal patch” and “iontophoretic patch” are used interchangeably herein to include integrated devices which allow for the administration of therapeutic compounds through the skin by using electrical current to promote the absorption of the compound from the patch through the skin of the subject. In particular, the therapeutic compounds of the present invention include rizatriptan in any form suitable for transdermal administration using iontophoresis. In one embodiment, the patch comprises electrical components, rizatriptan, and an adhesive backing layer.

The term “integrated” means completely self-contained and means patches which contain the drug, power source, and all other necessary components to deliver the drug ionotophoretically in a single wearable patch. The term “integrated”, thus, denotes patches which do not require a separate power source or controller. In some embodiments, the iontophoretic patch may be an integrated device, e.g., a wearable, self contained device which does not require a separate controller or power source. In some embodiments, the iontophoretic patch of the invention is not integrated, e.g., requires a separate controller, power source, etc, and may not necessarily be wearable.

One advantage of the iontophoretic patch of the present invention is that it is integrated and is wearable and lightweight. Examples of integrated iontophoretic patches may be found, for example, in PCT Application Publication No. WO 2007/120747, the entire contents of which are incorporated herein by reference. The device can include, for example, a release liner, saline and drug reservoirs, a double retaining ring overlayer (e.g., to contain the reservoirs), a power source and an outer protective layer.

The patches of the present invention may include, for example, a voltage source, one or more isolated anodes (e.g., containing zinc), a cathode or electrode (e.g., containing silver chloride), control transistors which may be in conjunction with parallel resistors, an exposed silver “wearbar” which connects the anodes to the circuitry. Activation of the patch is accomplished, for example, by application to the skin of a patient after removal of the release liner.

A variety of electrode compositions may be used in the patches of the invention. For example, a patch should include an active electrode, or electrodes, which do not significantly react with the rizatriptan or anti-migraine compound to form an insoluble salt. Zinc, or a metal with a reactivity similar to zinc is preferred. As illustrated, the electrodes, without limitation, may be zinc, zinc coated or contain zinc.

In some embodiments, the patch comprises an anode reservoir, a cathode reservoir and appropriate electrical circuitry. In some embodiments, an integrated patch comprises electrical components, rizatriptan, and an adhesive backing layer.

The backing layer can be any material known in the art for being suitable for such purposes. The backing layer is preferably flexible and suitable materials include without limitation, cellophane, cellulose acetate, ethylcellulose, plasticized vinylacetate-vinylchloride coploymers, polyethylene terephthalate, nylon, polyethylene, polypropylene, polyvinylidene chloride, coated flexible fibrous backings such as paper and cloth and aluminum foil. The adhesive material may be any material known in the art which is suitable for use in the iontophoretic patches of the invention.

In some embodiments, the patch comprises an electrode which does not significantly react with the rizatriptan or anti-migraine compound to form an insoluble salt. In some embodiments, the electrode is comprised of a metal with a reactivity similar to zinc. In some embodiments, the electrode includes silver, iron, aluminum, tin, copper, zinc, nickel, brass, metal alloys, conductive polymers, or coatings or mixtures thereof.

In some embodiments, the electrical circuitry of the patch comprises a battery which operates throughout use of the patch. In some embodiments, the battery is integrated into the patch and may be the main, if not sole, source of power of the patch.

The invention also includes embodiments in which said composition or the device of the invention further comprises an adsorbent material that is soaked or impregnated with said composition which is generally a liquid aqueous composition or hydrogel composition.

The adsorbent material which is soaked or impregnated with the aqueous or hydrogel composition(s) serves to keep said composition in place and, at the same time, to maintain the low-viscosity structure. Suitable adsorbent materials may be selected from fibrous pads, fabrics, sponges, tissues, non-woven or woven materials, felts or felt-like materials, etc.

According to some embodiments, the composition and/or patch of the present invention has adhesive properties, to ensure that the composition is maintained in direct and complete contact with the skin at the site of application during the whole time period of transdermal drug administration. Adhesiveness can be obtained by incorporating one or more adhesive polymers into said compositions. Adhesive polymers suitable for this purpose are generally known to the skilled person. In one embodiment, a polyamine or polyamine salt having adhesive properties is used as said adhesive polymer(s)

In some embodiments, the compositions and/or patches of the invention are self-adhesive. To render the compositions and/or patches self-adhesive, they may further contain one or more additives selected from the group of tackifiers which group includes, but is not limited to, hydrocarbon resins, rosin derivatives, glycols (such as glycerol, 1,3-butanediol, propylene glycol, polyethylene glycol), and succinic acid.

In some embodiments, the invention pertains, at least in part, to an iontophoretic transdermal patch for the delivery of rizatriptan or a salt thereof. The patch comprises an anode reservoir, a cathode reservoir and appropriate electrical circuitry for performing the methods of the invention.

In some embodiments, the patch has an iontophoretically active surface area of about 5 cm² or greater, 10 cm² or greater, 15 cm² or greater, 17.5 cm² or greater, 20 cm² or greater, 22.5 cm² or greater, 25 cm² or greater, 27.5 cm² or greater or 30 cm² or greater.

In some embodiments, when the iontophoretic pad is about 30 cm², the current density of the higher current density period is about 0.05 mA/cm² to about 0.25 mA/cm². The higher current densities allows for the quick delivery of therapeutically effective amounts of rizatriptan. Examples of currents which are used for the high current density periods include currents of about 2.5 mA to about 5 mA, e.g., about 3 mA to about 4 mA. In another further embodiment wherein the iontophoretic pad is about 5 cm², the current densities may be between about 0.25 mA/cm² to about 0.5 mA/cm², or about 0.3 mA/cm² to about 0.4 mA/cm².

The compositions of the invention may be formulated as hydrogels, e.g., flowable hydrogels, which include at least one gel-forming polymer, together with a gel-forming amount of water or aqueous solvent mixture.

The relative amounts of water and gel-forming components may be adjusted so as to obtain a hydrogel having solid or semi-solid consistency. However, the formulations of the present invention may also be formulated as liquids.

In some embodiments, the hydrogel compositions may comprise additional gel-forming polymers which may be selected, e.g., from polyacrylates or cellulose derivatives such as hydroxypropylmethyl cellulose, hydroxypropyl cellulose or hydroxyethyl cellulose.

The ionic strength can be adjusted by varying the proportion of water within the hydrogel. Thus, the ionic strength can be adjusted to optimize the efficacy of the iontophoretic process in each particular case.

In some embodiments, the reservoir includes a mixture that includes between about 0.1% and about 20%, between about 0.2% and about 10%, between about 2% and about 10%, between about 3% and about 5% rizatriptan or a salt thereof, or between about 0.1% and about 0.5% of rizatriptan or a salt thereof.

The reservoirs, e.g., which contain the rizatriptan or salt thereof, may be of any desired size. In some embodiments, the size of the reservoirs is chosen to provide a desired current density. In some embodiments, the reservoirs each have a surface area of about 1 cm², about 2 cm², about 3 cm², about 4 cm², about 5 cm², about 6 cm², about 7 cm², about 8 cm², about 9 cm², about 10 cm², about 11 cm², about 12 cm², about 13 cm², about 14 cm², about 15 cm², about 16 cm², about 17 cm², about 18 cm², about 19 cm², about 20 cm², about 21 cm², about 22 cm², about 23 cm², about 24 cm², about 25 cm², about 26 cm², about 27 cm², about 28 cm², about 29 cm², about 30 cm², about 31 cm², about 32 cm², about 33 cm², about 34 cm², about 35 cm², about 36 cm², about 37 cm², about 38 cm², about 39 cm², about 40 cm², about 41 cm², about 42 cm², about 43 cm², about 44 cm², about 45 cm², about 46 cm², about 47 cm², about 48 cm², about 49 cm², or about 50 cm² or greater.

In some embodiments, the reservoir is self-adhesive. The reservoir may also contain an additional tackifier, such as, but not limited, to hydrocarbon resins, rosin derivatives, glycols (e.g., glycerol, 1,3 butanediol, propylene glycol, polyethylene glycol), and succinic acid.

The term “solubility enhancer” includes compounds which increase the solubility of the rizatriptan in its vehicle. This can be achieved, for example, either through changing rizatriptan-vehicle interaction by introducing different excipients, or through changing the crystallinity of the rizatriptan. Examples of solubility enhancers include water diols, such as propylene glycol and glycerol; mono-alcohols, such as ethanol, propanol, and higher alcohols; DMSO; dimethylformamide; N,N-dimethylacetamide; 2-pyrrolidone; N-(2-hydroxyethyl)pyrrolidone, N-methylpyrrolidone, 1-dodecylazacycloheptan-2-one and other n-substituted-alkyl-azacycloalkyl-2-ones.

The term “flux enhancer” includes compounds which increase the ionic flux of rizatriptan, i.e., so as to increase the rate at which the rizatriptan permeates through the skin and enters the bloodstream. The enhanced flux effected through the use of such enhancers can be observed, for example, by measuring the rate of diffusion of the rizatriptan through animal or human skin using a diffusion cell apparatus. In some embodiments, the flux enhancer acts to increase the flux of the rizatriptan across the skin of the subject when utilized with an iontophoretic patch of the present invention.

Examples of flux enhancers include, but are not limited to, dimethylsulfoxide (DMSO), N,N-dimethylacetamide (DMA), decylmethylsulfoxide (C₁₀ MSO), polyethylene glycol monolaurate (PEGML), propylene glycol (PG), PGML, glycerol monolaurate (GML), lecithin, the 1-substituted azacycloheptan-2-ones, particularly 1-n-dodecylcyclazacycloheptan-2-one, alcohols, and the like. The flux enhancer may also be a vegetable oil such as, for example, safflower oil, cotton seed oil and corn oil.

In some embodiments, the flux enhancer is a flux enhancer which is uncharged at neutral pH. In some embodiments, the flux enhancer is selected from lauric acid, polyoxyethylene (4) lauryl ether, sorbitan laurate and mixtures thereof.

In addition, other agents may be used to enhance the solubility of the polyamine or hydrogel. Examples of such solubilizing agents include, but are not limited to, fatty acids, e.g., C₆-C₁₄ saturated fatty acids. Examples of saturated fatty acids include hexanoic, decanoic, myristic, palmitic, lauric and caprylic acids. In some embodiments, the fatty acid is lauric acid and is present in amounts between about 0.1% and about 10%, between about 0.2% and about 9.5%, between about 0.3% and about 9.0%, between about 0.4% and about 8.5%, between about 0.5% and about 8.0%, between about 1.0% and about 7.0%, between about 1.5% and about 6.0%, between about 2.0% and about 5.0%, between about 3.0% and about 4.0% and about 3.40%.

In some embodiments, the iontophoretic devices of the present teachings include an antimicrobial agent, e.g., in one or both of the reserviors. The term “antimicrobial agent” includes agents which prevent the growth of microbials in the patch. Examples of antimicrobials include, but are not limited to, salts of chlorhexidine, such as iodopropynyl butylcarbamate, diazolidinyl urea, chlorhexidene digluconate, chlorhexidene acetate, chlorhexidene isethionate, and chlorhexidene hydrochloride. Other cationic antimicrobial agents may also be used, such as benzalkonium chloride, benzethonium chloride, triclocarbon, polyhexamethylene biguanide, cetylpyridium chloride, methyl and benzothonium chloride. Other antimicrobial agents include, but are not limited to: halogenated phenolic compounds, such as 2,4,4′,-trichloro-2-hydroxy diphenyl ether (Triclosan); parachlorometa xylenol (PCMX); and short chain alcohols, such as ethanol, propanol, and the like. Other examples of antimicrobial agents include methyl para-hydroxybenzoate or methyl 4-hydroxy benzoate.

In some embodiments, the compositions of the invention comprise between about 0.01% and about 1.0%, between about 0.05% and about 0.5%, between about 0.07% and about 0.4%, between about 0.08% and about 0.3%, between about 0.09% and about 0.2%, and about 0.10% methyl para-hydroxybenzoate.

In some embodiments, the solution has a pH of about 3 to about 8, about 5.5 to about 7, or about 6. In some embodiments, the pH of solution is selected such that usage of the patch does not substantially affect the pH of the skin. In some embodiments, the pH of the skin changes about ±4.0 or less, about ±3.5 or less, about ±3.0 or less, about ±2.5 or less, about ±2.0 or less, about ±1.5 or less, about ±1.0 or less, or about ±0.5 of less.

Generally, the compositions of the present invention can be manufactured by conventional methods. The compositions of the present invention are obtainable by dissolving or dispersing the various ingredients in water or an aqueous solvent mixture. The resulting mixture may then be spread on a flat surface or poured into moulds or extruded, and then allowed to solidify to obtain hydrogel compositions having the desired shape. During these process steps, or after solidification, the composition may be combined with additional components as required to produce the final product, generally a pharmaceutical administration form.

However, various alternative methods for manufacturing the compositions of the present invention may be used, as will be readily realized by the skilled person.

The present invention further encompasses the use of the above-described composition(s) as an integral component of a transdermal patch. Preferably, such composition is incorporated into said patch during manufacture, to form the active substance reservoir of the patch. Further, the present invention encompasses the use of the above-described composition(s) as an integral component of an iontophoretic patch, for example, as an anodic reservoir of the patch. Such composition may be incorporated into the iontophoretic patch during manufacture, to form the anodic reservoir of the patch. The above-mentioned administration forms are obtainable by manufacturing methods generally known in the art.

In some embodiments, the patches of the invention are light weight. An iontophoretic patch according to the invention may weigh less than about 0.5 lbs, less than about 0.4 lbs, less than about 0.3 lbs, less than about 0.2 lbs, less than about 0.1 lbs, or less than about 0.05 lbs. Alternatively, a patch according to the invention may weigh less than about 500 grams, less than about 400 grams, less than about 300 grams, less than about 200 grams, less than about 100 grams, less than about 50 grams, less than about 25 grams, or even less than about 10 grams.

Rizatriptan may be advantageously stored separated from electronic components of the patch. The term “separated” refers to patches where the electronic portions of the patch do not come into contact with the drug before the patch is activated.

In some embodiments, the patches have a long “shelf life” and can be stored until the subject is suffering or is at risk of suffering from a rizatriptan responsive state. Without wishing to be bound by any particular theory, it is believed that this long shelf-life may be due to the ability to keep the rizatriptan or salt thereof separate from the electronic elements of the patch. The term “shelf life” includes the period of time that the complete patch can rest unused in ambient temperature and moisture levels and still be used to perform its intended function, e.g., administer the desired compounds to treat a subject. In certain embodiments, the shelf life of the patch of the invention is at least 1 month or longer, at least 2 months or longer, at least 3 months or longer, at least 4 months or longer, at least 5 months or longer, at least 6 months or longer, at least 7 months or longer, at least 8 months or longer, at least 9 months or longer, at least 10 months or longer, at least 11 months or longer, at least one year or longer, at least 18 months or longer, or at least 2 years or longer.

### Example I

### Permeation Through Dialysis Membrane

This example shows that zolmitriptan and rizatriptan have greater permeation through dialysis membrane due to iontophoresis than sumatriptan.

### Materials and Methods

Ionotophoresis side-by side diffusion cells with 9 mm diameter opening were obtained from PermeGear. The current clamps with PC control were provided by NuPathe, Inc. The temperature controller was from Henke. 2.0 mm diameter Zinc and Silver were obtained from Alpha Aesar. Sumatriptan, Zolmitriptan and Rizatriptan wore provided by NuPathe. Sodium chloride was obtained from Fisher Scientific,

A donor solution of 4% sumatriptan succinate in 0.9% NaCl was prepared. The receiver solution was 0.9% Sodium chloride in water. A Millipore 1000 mw cutoff membrane were used as the membrane. The glossy side of the membrane faced the donor compartment. The volume of the donor and receiver solution was 3.0 ml. 0.5 mm zinc wire was placed in the donor compartment and 2.0 mm silver wire was placed in the receiver compartment. The temperature of the apparatus was maintained at 33° C.

Ketoprofen was obtained from Spectrum Chemical. A 2% ketoprofen donor solution was made by dissolving in 0.1 N NaOH.

Three cells were clamped at 2 mAmps/cm² (1.27 mAmp/cell), 3 cells were clamped at 2 mAmp/cm² with the electrodes reversed, 3 cells (the control cells) were not clamped.

At each sampling point 2 ml was withdrawn from the receiver compartment and was replaced with 0.9% Sodium Chloride. Each sample was diluted using 0.9% NaCl to be within the standard curve. Absorbance was measured using a UV spectrophotometer with a 1 cm quartz cuvette at 282 nm.

### Results

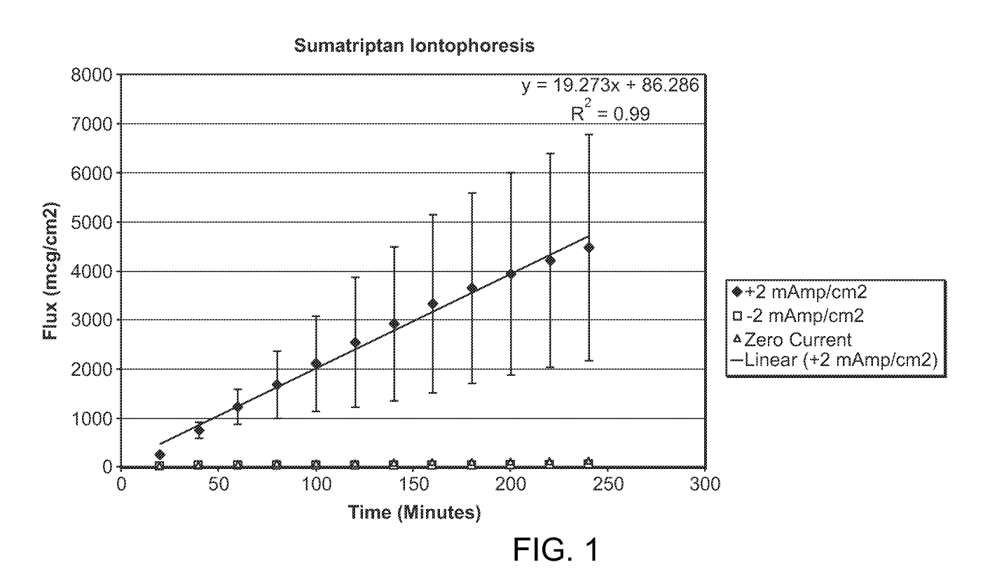

A graph depicting data regarding permeation through the dialysis membrane is shown in FIG. 1. As the data shows, the iontophoresis of sumatriptan is much faster than the control and the iontophoresis conducted with reversed electrodes. Additionally, the passive diffusion (zero current) was then compared to the diffusion with the electrodes switched. It is noted that the passive diffusion and the electrode switch diffusion were comparable and that the electrode-switched cells approached equilibrium. An additional plot of C/Cmax suggested that the flux approaches equilibrium, possibly because the chemical potential caused by the high concentration of the drug is running into an opposite force of electrical potential.

The ketoprofen standard curve showed that ketoprofen UV absorbance was linear over a wide range of concentrations. The data also showed that the reversed electrodes provides for the greatest flux of ketoprofen. This is expected because ketoprofen is anionic when in solution, therefore, the greatest flux is expected when the cathode is in the donor compartment and the anode is in the receiver compartment.

Unlike sumatriptan, the flux of ketoprofen is substantial for passive diffusion. This would suggest that the flux due to passive diffusion is great enough to produce a dosage form without electrical supplementation. The flux of ketoprofen utilizing the standard configuration (anode in the donor and cathode in the receiver) was less than the passive diffusion. The ketoprofen did not reach equilibrium during the experiment, possibly because the flux due to passive diffusion is much greater than the flux due to iontophoresis.

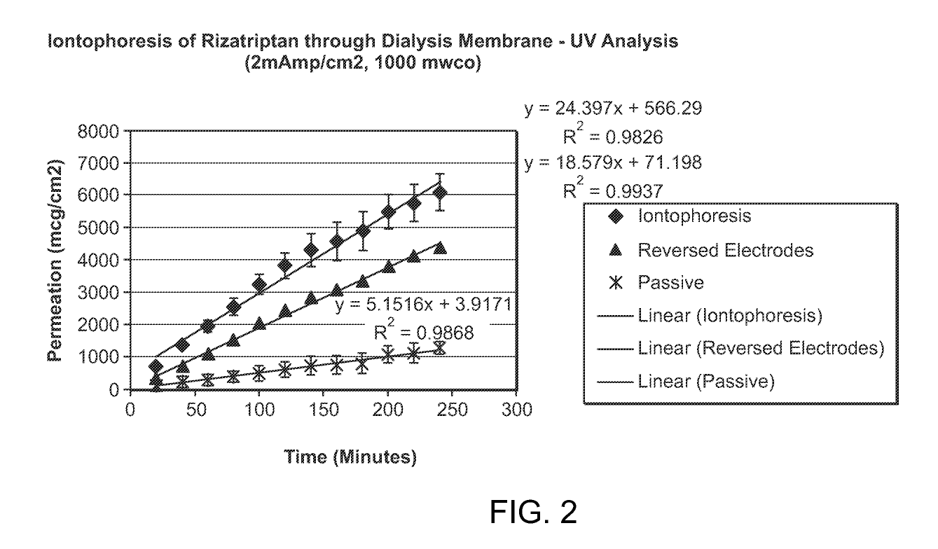

FIG. 2 is a graph depicting both the iontophoresis of rizatriptan and the reversed-electrodes iontophoresis data. This figure shows that both had great flux compared to the passive diffusion. This may be due to the fact that the UV assay is actually measuring both benzoate and rizatriptan instead of only rizatriptan. To confirm this data the samples were analyzed via HPLC to establish the actual rizatriptan flux.

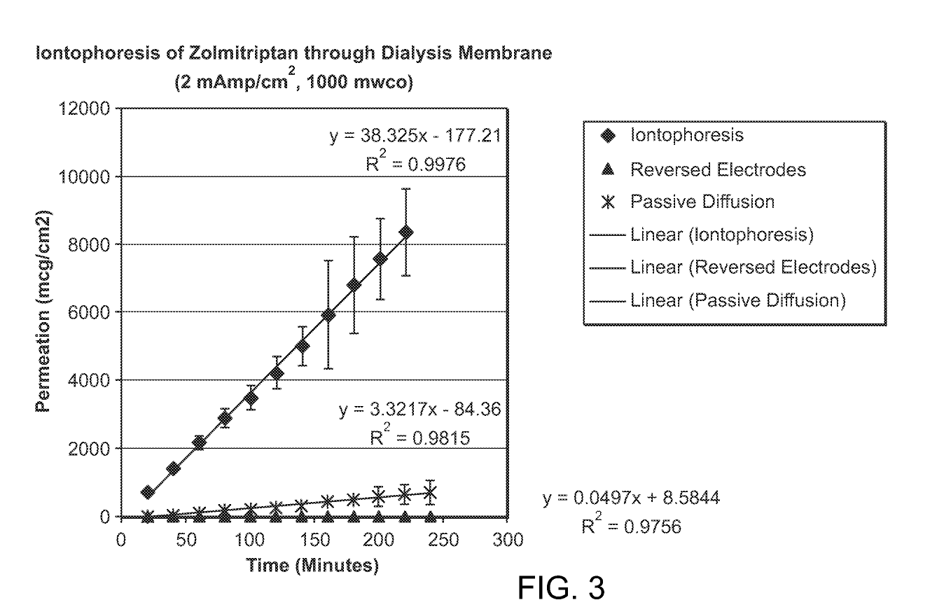

FIG. 3 is a graph depicting the permeation data for Zolmitriptan. The data indicates that both iontophoretic and passive diffusion for Zolmitriptan show greater permeation compared to sumatriptan. Additionally, the iontophoresis permeation is approximately 11 times greater than the permeation due to passive diffusion.

The ratio of iontophoresis flux to passive diffusion also suggests some unique properties of each candidate:
1. Sumatriptan had the highest ratio of iontophoresis to passive diffusion at 43, possibly because sumatriptan had low passive diffusion through the membrane.
2. Zolmitriptan had the next highest ratio of 11.5 (the highest passive diffusion of any drug candidate tested). The passive diffusion of zolmitriptan was greater than the iontophoresis permeation of sumatriptan.
3. Although zolmitriptan had a high passive diffusion component, the flux was much less when the electrodes were reversed compared to the passive diffusion. (3.32 mcg/cm²/min for passive diffusion and 0.05 mcg/cm²/min for reversed electrodes). Thus, iontophoresis is still a great driving force for permeation of zolmitriptan.
4. Zolmitriptan has the potential to reach therapeutic drug concentrations at a much faster rate compared to sumatriptan.

Rizatriptan was assayed using an HPLC method. The mobile phase was 10 mM potassium phosphate monobasic:Trimethylamine:Methanol (85.5:9.5:5 v/v). Potassium phosphate monobasic solution and TEA were mixed and the pH of solution was adjusted to pH 5.5. Methanol was then added to this solution. A HP 1050 HPLC with DAD with a Waters 4.6×15 mm ODS column was used. The flow was 1.0 ml/min. The system integrated peaks detected at 225 nm Rizatriptan had a retention time of 3.2 minutes and benzoic acid had a retention time of 8.5 minutes.

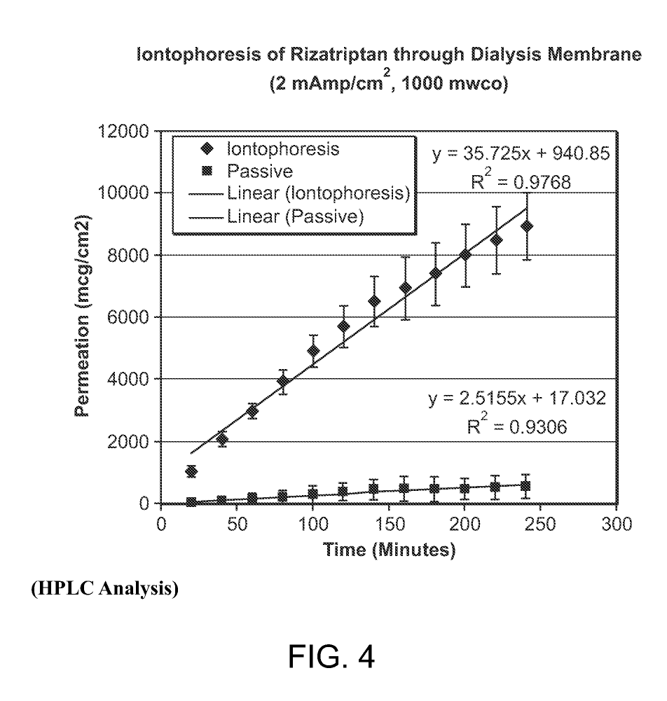

FIG. 4 is a graph depicting the HPLC results for the analysis of rizatriptan through the dialysis membrane shows much greater permeation of rizatriptan by iontophoresis than for passive diffusion. The flux was 35.73 mcg/cm²/min for iontophoresis compared to 2 mcg/cm²/min for passive diffusion. The reversed electrodes show no detectable permeation.

The ratio of iontophoresis flux to passive diffusion is 14.2 for rizatriptan compared to 11.5 for zolmitriptan and 43.4 for sumatriptan (see Table 1). Thus, the rank order of iontophoresis flux is Rizatriptan=Zolmitriptan>Sumatriptan. The passive flux has a similar rank order of Zolmitriptan>Rizatriptan>>Sumatriptan.

TABLE 1
Flux of Drugs through 1000 mwco
dialysis membrane in mcg/cm²/min

| | | | Reversed | |
|---|---|---|---|---|
| | | | Electrodes | Ratio of |
| | Iontophoresis | Passive | (Current | Iontophoresis |
| | (2 | diffusion | reversed at 2 | to Passive |
| Drug | mAmp/cm²) | (No current) | mAmp/cm²) | Diffusion |
| Sumatriptan | 25.2 | 0.58 | 0.11 | 43.4 |
| Ketoprofen¹ | 8.24 | 7.41 | 11.66 | 1.6 |
| Rizatriptan | 24.40 | 5.15 | 18.80 | 4.7 |
| (UV) | | | | |
| Rizatriptan | 35.73 | 2.52 | ~0 | 14.2 |
| (HPLC) | | | | |
| Zolmitriptan | 38.33 | 3.32 | 0.05 | 11.5 |

¹Ketoprofen is anionic. Reversed electrode flux should be greater than Iontophoresis.

Additional analysis of the charge and molecular weight properties indicated that rizatriptan has the greatest potential charge and sumatriptan has the lowest. (See Table 2) This did not result in the greatest permeation for rizatriptan in these experiments. This is because the diffusion and permeation through a hydrophilic membrane are dependent on molecular weight, radius of the molecule and the ionic drag the molecule has with the membrane.

TABLE 2
Iontophoresis Properties

| | Molecular | Ionizable | MW/charge |
|---|---|---|---|
| Drug | weight | Nitrogens | Ratio |
| Rizatriptan | 269.4 | 3 | 89.8 |
| Sumatriptan | 295.4 | 2 | 147.7 |
| Zolmitrlptan | 278.36 | 3 | 92.8 |

### Example II

### Permeation of Compound Through Human Cadaver Skin

The iontophoresis of the drug candidates through skin may be completely different. For example, although charge and molecular weight are important parameters for diffusion through skin, partition coefficient is equally important. Additionally, charge would add a significant drag to the permeation of the molecule through the hydrophobic regions of the stratum corneum. This example shows that rizatriptan has a significantly greater iontophoretic permeation through human cadaver skin compared to both sumatriptan and zolmitriptan.

Human cadaver skin was obtained from Allosource. The skin was maintained at −70° C. prior to use. A portion of skin was thawed in 0.9% NaCl solution for 30 minutes. The skin was towel dried and cut into 2 cm square sections. The skin was mounted with the epidermis side toward the donor chamber in the Permeagear 0.9 cm diameter side-by-side diffusion cells.

A solution of 2% zolmitriptan was made by dissolving zolmitriptan in 0.1 N HCl. A 4% sumatriptan solution was made by dissolving sumatriptan in 0.1% NaCl. The 4% rizatriptan solution was made by dissolving the rizatriptan in 0.1% NaCl

Three cells were dosed with 2% zolmitriptan, three cells were dosed with 4% rizatriptan, and two cells were dosed with 4% sumatriptan. Normal saline solution was used as the receiver solution. The cells were clamped at 1.27 mAmp (2 mAmp/cm²). 2 ml of each receiver compartment was sampled every 30 minutes and replaced with normal saline solution. One cell of the rizatriptan iontophoresis and one cell of sumatriptan iontophoresis were lost during the study due to holes in the skin.

Each 2 ml sample was diluted 1 to 100 with water. Zolmitriptan and sumatriptan were assayed by UV at 222 nm and 282 nm respectively. Rizatriptan was assay by HPLC as previously described.

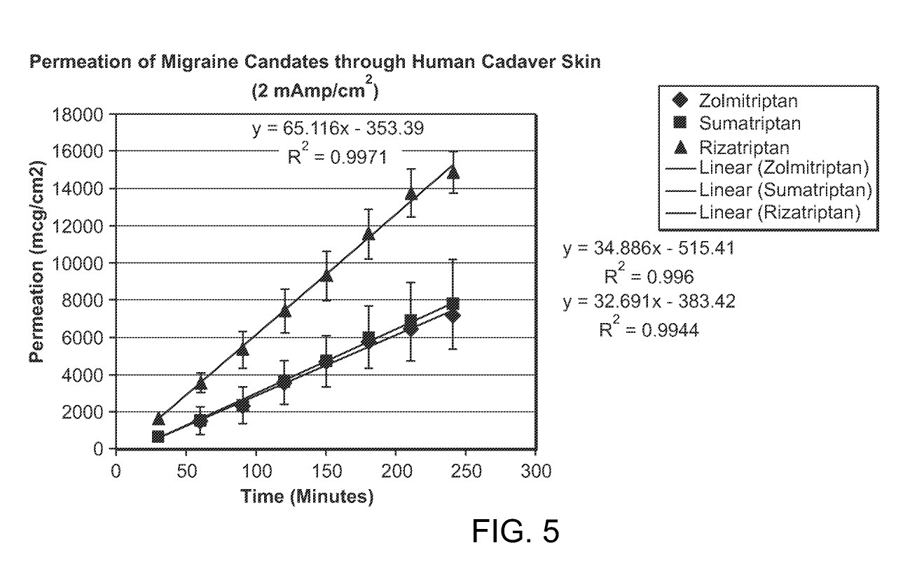

The results are shown in FIG. 5. As indicated in this figure, rizatriptan has greater skin permeation than sumatriptan and zolmitriptan. The permeation of zolmitriptan by iontophoresis was very comparable to the permeation of sumatriptan via iontophoresis.

Without wishing to be bound by any particular theory, it is believed that the increased rizatriptan flux may be caused by one or more of the following factors:
1. Rizatriptan has a high number of possible ionizable nitrogens
2. Rizatriptan has ions that are well shielded because all the ionizable nitrogens are tertiary or ring nitrogens.
3. The counter ion for rizatriptan is benzoic acid, which may enhance permeation.

### Example III

### Permeation of Formulated Product Through Human Cadaver Skin

Continued work on a formulated product of rizatriptan and zolmitriptan appeared necessary at this point. This example shows that formulations which include laureth-4 (Brij 30) shows enhanced iontophoretic permeation of rizatriptan. Accordingly, low molecular weight nonionic surfactants hold promise as flux enhancers. The results from these studies suggest that a dose of rizatriptan (5 mg) can be delivered in about 45 minutes.

Brij 30 (Laureth-4) and adipic acid were obtained from Fisher Scientific. Eudragit E*PO was obtained from Evonik. Three formulations were made as shown in Table 3.

Human cadaver skin was thawed in 0.9% NaCl for 30 minutes and patted dry. The skin was cut into 2 cm square sections. The epidermis was placed towards the donor compartment of a 0.9 cm radius PermeGear side-by-side diffusion cell. The donor gel was placed in the donor compartment and 3 ml of normal saline solution was placed in the receiver compartment. Three cells were dosed with zolmitriptan with 3.4% lauric acid, three cells were dosed with rizatriptan with 3.4% lauric acid and two cells were dosed with rizatriptan with 3.4% Brij 30.

The cells were clamped at 1.27 mAmp (2 mAmp/cm²). Two ml of each receiver compartment was sampled every 30 minutes and replaced with normal saline solution.

Each 2 ml sample was diluted 1 to 100 with water. Zolmitriptan and sumatriptan were assayed by UV at 222 nm Rizatriptan was assayed by HPLC as previously described.

TABLE 3
Drug Formulations for Iontophoresis.

| | Drug | Eudragit | flux | Adipic |
|---|---|---|---|---|
| Formulation | concentration | E PO | enhancer | acid |
| I | 2% Zolmitriptan | 10% | 3.4% Lauric Acid | 0.3% |
| II | 4% Rizatriptan | 10% | 3.4% Lauric Acid | 0.3% |
| III | 4% Rizatriptan | 10% | 3.4% Brij 30 | 0.3% |

Formulations adjusted to pH 5.0 using ortho-phosphonic acid.

The resulting iontophoresis shows that rizatriptan with lauric acid had a greater flux of 25.4 mcg/cm²/min compared to 22.9 mcg/cm²/min for zolmitriptan with lauric acid. The lag time for rizatriptan was 9 minutes compared to 20 minutes for zolmitriptan.

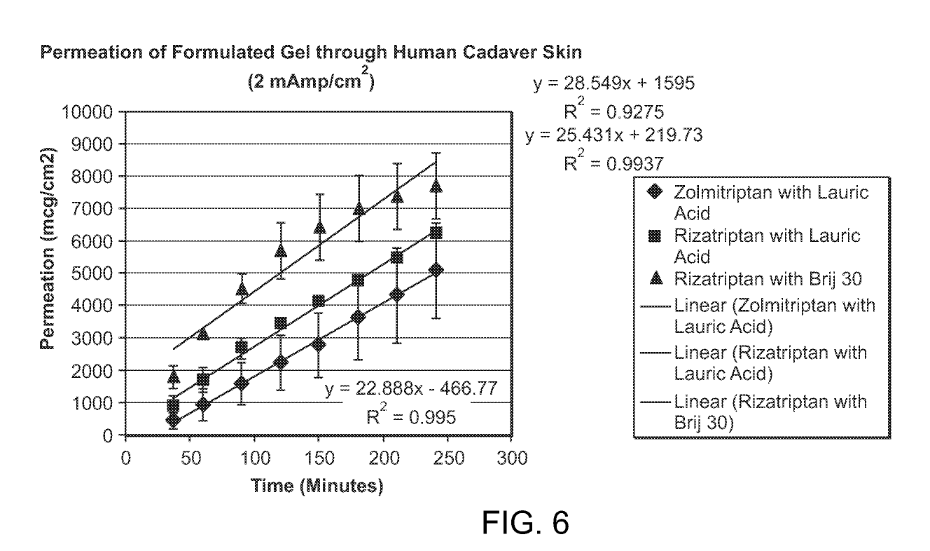

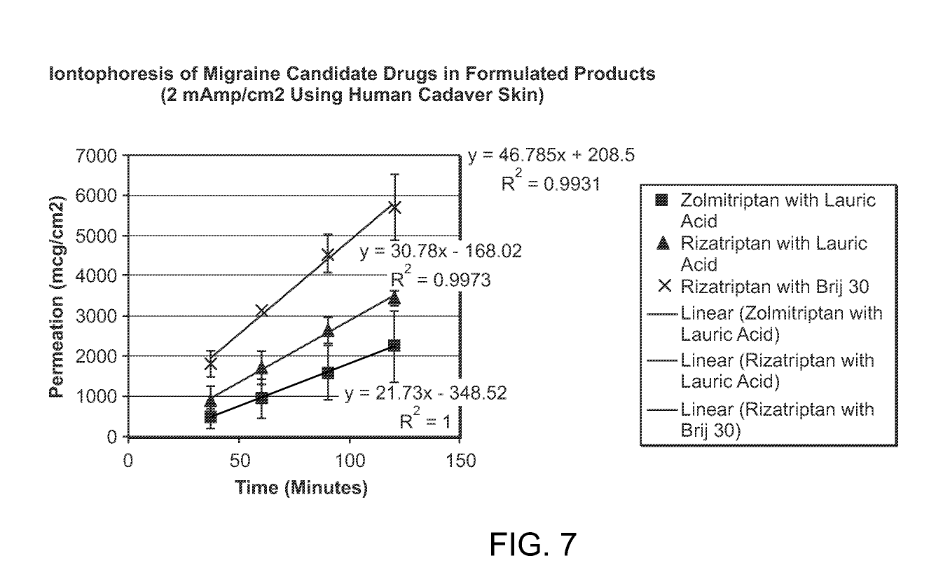

Rizatriptan with Brij 30 had the greatest flux at 28.5 mcg/cm²/min and a burst time of 55 minutes. Without wishing to be bound by any particular theory, it is believed that this burst effect was caused by an apparent deceleration in the flux over time, which corresponds to an increase in voltage observed toward the end of the experiment. (See FIGS. 6 and 7).

Such a phenomenon may be caused, for example, by one or both of the following factors:
1. An increase in the flux of the counter ions which resulted in a proportional increase in the voltage to maintain the flux.
2. A back potential created by chloride flux, resulting in a current that could not be maintained.

Analysis of the first 4 time points of the Rizatriptan with Brij 30 shows that the initial flux rate was 46.8 mcg/cm²/min. The rizatriptan with lauric acid shows a comparable rate of 30.8 mcg/cm²/min suggesting that there is little deceleration when lauric acid is used. One reason that the Brij 30 may have worked so well is that it is uncharged at all pHs. Lauric acid is significantly ionized at neutral pH based on a pKa of around 4.9, which, it is believed, may decrease the efficiency of the enhancer. Iontophoresis would also decrease the amount of lauric acid available for enhancement, especially if the pH of the solution was below 5.5. Either cause would result in improved enhancement with Brij 30 compared to lauric acid. Span 20 is also considered for future experiments, as it has similar enhancement properties to Brij 30 but may result in less irritation.

Additional analysis of the permeation data was conducted to find the amount of time to dose 5 mg of drug based on reasonable patch areas, and is shown in Table 4. The analysis shows that both zolmitriptan and rizatriptan would deliver a 5 mg dose in less than 15 minutes. However, as was observed by the previous permeation data, zolmitriptan would need a total of 43 minutes when the lag-time is considered compared to 19 minutes for rizatriptan when the lag time is considered.

TABLE 4
Dosing time based on skin Permeation (based on 2 mAmp/cm²)

| | Time to deliver 5 mg (Minutes) | | |
|---|---|---|---|
| | Area | | |
| Rate | 10 cm² | 20 cm² | 40 cm² |
| 46.8 | 10.7 | 5.3 | 2.7 |
| 30.7 | 16.3 | 8.1 | 4.1 |
| 21.7 | 23.0 | 11.5 | 5.8 |

### Example IV

### Oxidation Stability for Rizatriptan

Iontophoresis places an oxidative pressure on drug molecules. In this example, the objective was to evaluate whether there is rizatriptan degradation due to this oxidative pressure.

A drug stock solution was made by dissolving 2.8 mg of rizatriptan benzoate in 0.9% NaCl to a volume of 50 mL. The working standard was prepared by diluting 0.5 ml of the stock solution to 10 ml with 0.9% NaCl. This produced a final concentration of 2.8 mcg/ml, for the working standard.

A solution was made by dissolving 0.5000 g of rizatriptan benzoate in 0.9% NaCl to a final volume of 50 mL. A 1 ml sample of this solution was kept for future assay. The remaining solution was placed in a beaker covered with parafilm Zinc wire (Cathode) and silver wire (anode) were passed through the parafilm and placed in the drug solution. Current of 2 mA was passed through the solution. Samples of 1 mL each were withdrawn every 30 minutes for 5.5 hours.

A 0.1 mL volume from each sample was diluted with 15 mL of 0.9% NaCl. A 1 ml volume of this dilution was then further diluted to 10 ml using 0.9% NaCl. Similarly, a 0.1 ml volume of the initial solution was diluted with 15 mL of 0.9% NaCl. A 1 ml volume of this dilution was then diluted to 10 ml with 0.9% NaCl.

Table 5 shows the concentration data at each time. There is a general trend in that the later time-points have a lower concentration compared to the initial solution. Regression analysis shows that the slope is significant when all data is used. The approximate rate of degradation was 16 mcg/ml/min. This results in approximately 7% degradation during the 5.5 hour exposure.

Upon additional analysis of the data it was observed that the final two time points are significantly lower than the remaining data. When the regression analysis was conducted without the last two time points the slope was not significantly different than zero. Although the concentration does drop during the first 4 hours of the experiment, degradation of the drug does not become significant until after 4 hours of exposure.

Rizatriptan does show potential for degradation due to the oxidation/reduction pressure placed on the drug during iontophoresis; however, the rate of degradation is slow. The use of antioxidants in the final formulation of an iontophoretic patch may help to slow or stop this degradation

TABLE 6
Rizatriptan concentration after
exposure to 2 mAmp current.

| Time(min) | Concentration(mg/mL) |
|---|---|
| 0 | 10.000 |
| 30 | 10.041 |
| 60 | 9.829 |
| 90 | 9.909 |
| 120 | 9.782 |
| 150 | 9.942 |
| 180 | 9.875 |
| 210 | 9.801 |
| 240 | 9.758 |
| 270 | 9.963 |
| 300 | 9.216 |
| 330 | 9.389 |

### EQUIVALENTS

Those skilled in the art will recognize, or be able to ascertain using no more than routine experimentation, numerous equivalents to the specific procedures described herein. Such equivalents are considered to be within the scope of the present invention and are covered by the following claims. The contents of all references, patents, and patent applications cited throughout this application are hereby incorporated by reference. The appropriate components, processes, and methods of those patents, applications and other documents may be selected for the present invention and embodiments thereof.

What is claimed is:

1. A method for treating a subject for a rizatriptan responsive state, comprising transdermally administering to the subject an effective amount of rizatriptan or a salt thereof in less than 45 minutes using an integrated iontophoretic patch, wherein the rizatriptan or salt thereof is formulated in a flowable hydrogel and wherein said patch uses a current density selected such that said current does not substantially irritate said subject's skin.

2. The method according to claim 1, wherein an effective amount of rizatriptan or a salt thereof is administered to the subject in less than 30 minutes.

3. The method according to any one of the preceding claims, wherein the patch provides rizatriptan at an iontophoretic flux rate of about 22.9 mcg/cm²/min or greater across the subject's skin.

4. The method according to any one of the preceding claims, wherein the patch provides rizatriptan at an iontophoretic flux rate of about 28.5 mcg/cm²/min or greater across the subject's skin.

5. The method according to any of the preceding claims, wherein said patch uses an average current density of 0.20 mA/cm² or less for a significant portion of delivery time of the rizatriptan or salt thereof.

6. The method according to any of the preceding claims, wherein said patch uses an average current density of 0.15 mA/cm² or less for a significant portion of delivery time of the rizatriptan or salt thereof.

7. The method according to any of the preceding claims, wherein said patch uses an average current density of 0.10 mA/cm² or less for a significant portion of delivery time of the rizatriptan or salt thereof.

8. The method according to any of the preceding claims, wherein said patch uses an average current density of 0.05 mA/cm² or less for a significant portion of delivery time of the rizatriptan or salt thereof.

9. The method according to any of the preceding claims, wherein the patch provides an uninterrupted two-stage patterned delivery sequence wherein current densities average between about 0.05 and about 0.40 mA/cm² during a significant portion of a first stage followed by a second stage delivery wherein current densities average between about 0.01 and about 0.40 mA/cm² during a significant portion of the second stage, to provide a waveform delivery pattern in which a therapeutically effective dosage level is reached in a subject in less than about one hour and a maintenance level is continued for one or more hours.

10. The method according to any one of the preceding claims, wherein usage of the patch to deliver a steady state concentration of said rizatriptan results in a mean skin erythema score of 1.00 or less immediately after patch removal.

11. The method according to any one of the preceding claims, wherein usage of the patch to deliver a steady state concentration of said rizatriptan results in a mean skin erythema score of 0.50 or less immediately after patch removal.

12. The method according to any one of the preceding claims, wherein usage of the patch to deliver a steady state concentration of said rizatriptan results in a mean skin erythema score of zero immediately after patch removal.

13. The method according to any one of the preceding claims, wherein said hydrogel comprises a flux enhancer.

14. The method according to claim 13, wherein the flux enhancer is a flux enhancer which is uncharged at neutral pH.

15. The method according to claim 13, wherein the flux enhancer is at least one enhancer selected from lauric acid, polyoxyethylene (4) lauryl ether and mixtures thereof.

16. The method according to claim 13, wherein said hydrogel comprises at least about 3.4% polyoxyethylene (4) lauryl ether.

17. The method according to claim 1, wherein the effective amount is a concentration of about 20 ng/mL or less in the subject's blood.

18. The method according to claim 1, wherein the patch is able to maintain an effective steady state concentration of the rizatriptan or salt thereof in the subject's blood for at least about an hour.

19. An integrated iontophoretic transdermal patch for the delivery of rizatriptan or a salt thereof to a subject in need thereof, comprising
a controller to deliver at least a portion of said rizatriptan or salt thereof to the subject by driving an electrotransport current through an animal body surface using a controllable power supply;
an electrode which does not form an insoluble salt of the rizatriptan or salt thereof; and
a composition comprising rizatriptan or a salt thereof formulated in a flowable hydrogel;
wherein the patch delivers an effective amount of rizatriptan or a salt thereof to the subject in less than 45 minutes.

20. The patch according to claim 19, wherein the patch provides rizatriptan at an iontophoretic flux rate of about 22.9 mcg/cm²/min or greater across the subject's skin.

21. The patch according to any one of claims 19-20, wherein the patch provides rizatriptan at an iontophoretic flux rate of about 28.5 mcg/cm²/min or greater across the subject's skin.

22. The patch according to any one of claims 19-21, wherein the controller provides an uninterrupted two-stage patterned delivery sequence wherein current densities average between about 0.05 and about 0.20 mA/cm² during a significant portion of a first stage followed by a second stage delivery wherein current densities average between about 0.01 and about 0.20 mA/cm² during a significant portion of the second stage, to provide a waveform delivery pattern in which a therapeutically effective dosage level is reached in a subject in less than about one hour and a maintenance level is continued for one or more hours.

23. The patch according to any one of claims 19-22, wherein usage of the patch to deliver a steady state concentration of the rizatriptan or salt thereof results in a mean skin erythema score of 1.00 or less immediately after patch removal.

24. The patch according to any one of claims 19-23, wherein said hydrogel contains a flux enhancer.

25. The patch according to claim 24, wherein the flux enhancer is a flux enhancer which is uncharged at neutral pH.

26. The patch according to claim 24, wherein the flux enhancer is at least one enhancer selected from lauric acid, polyoxyethylene (4) lauryl ether, sorbitan laurate and mixtures thereof.

27. The patch according to claim 24, comprising at least 4% rizatriptan and at least 3.4% polyoxyethylene (4) lauryl ether.
